(19) United States
(12) Patent Application Publication
(10) Pub. No.: US 2008/0228065 A1
(43) Pub. Date: Sep. 18, 2008

(54) System and Method for Registration of Localization and Imaging Systems for Navigational Control of Medical Devices

(76) Inventors: Raju R. Viswanathan, St. Louis, MO (US); Walter M. Blume, St. Louis, MO (US); Meir Bartal, Zichron Yaacov (IL); Assaf Rubissa, Misgav (IL)

Correspondence Address: HARNESS, DICKEY, & PIERCE, P.L.C, 7700 Bonhomme, Suite 400, ST. LOUIS, MO, 63105, US

(21) Appl. No.: 11/685,684

(22) Filed: Mar. 13, 2007

(51) Int. Cl. A61B 6/02

(52) U.S. Cl. 600/424; 600/426

(57) ABSTRACT

A system and method are provided for obtaining registration of a localization system's three-dimensional coordinates for a plurality of markers having known locations to the two-dimensional coordinates for the plurality of reference markers obtained from a single X-ray image. The method for registering a navigational system with a localization system having a plurality of reference markers includes determining the three-dimensional coordinates of the locations of a plurality of reference markers within the localization coordinate system, and providing a single two-dimensional X-ray image that includes a two dimensional location of the plurality of markers. The method further includes determining a coordinate transformation for obtaining a best fit registration of the localization system's three-dimensional coordinates to the two-dimensional coordinates obtained from the single X-ray image, for at least one reference marker location.

### FIELD

The present disclosure relates to the navigation of medical devices in the presence of radiopaque materials, and in particular to a method of navigating a medical device for the delivery of radiopaque materials.

### BACKGROUND

The statements in this section merely provide background information related to the present disclosure and may not constitute prior art.

Interventional medicine technologies have been applied to manipulation of instruments which contact tissues during surgical procedures, making these procedures more precise, repeatable and less dependent of the device manipulation skills of the physician. Some presently available interventional medical systems for directing and manipulating the distal tip of the medical device by remote actuation use computer assisted navigation and an imaging system for providing imaging of the catheter and blood vessels and tissues. The system may also be configured to include a localization system for determining the position of the catheter or medical device in the localization system's own frame of reference translatable to the displayed image of the imaging system. In the absence of an explicit link between the localization system and the imaging and navigation systems, the localized device data is not readily available to control navigation of the device by computer-controlled means.

### SUMMARY

The present disclosure relates to systems and methods for providing registration of a localization system with an imaging system associated with a navigational control system for a medical device.

In one aspect of the present disclosure, a system and method for providing registration of a localization system with an imaging system associated with a navigational control system are provided. One embodiment of a system according to the principles of the present invention includes an X-ray imaging system, and a localization system having a location pad. The localization system's location pad is preferably capable of sensing electromagnetic signals transmitted and received between the medical device and the location pad, which signals are processed by the localization system for determining the position of the distal end of the medical device within a subject's body. The location pad is positioned relative to a patient table, and includes at least two plates removably inserted within the location pad, which plates include a plurality of reference markers that are visible within a single X-ray imaging plane.

In another aspect of the present disclosure, one or more methods are provided for obtaining registration of a localization system's three-dimensional coordinates for a plurality of markers having known locations to the two-dimensional coordinates for the plurality of reference markers obtained from a single X-ray image. In one embodiment, a method for registering an imaging or navigational system with a localization system having a plurality of reference markers with known coordinates comprises determining the three-dimensional coordinates of the locations of a plurality of reference markers within the localization coordinate system, and providing a single two-dimensional X-ray image that includes a two dimensional location of the plurality of markers. The method further comprises determining a coordinate transformation for obtaining a best fit registration of the localization system's three-dimensional coordinates to the two-dimensional coordinates obtained from the single X-ray image, for at least one reference marker location.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1 is a perspective view of a location pad of a localization system, having a marker plate inserted therein;

FIG. 2 is a cross-sectional view of a location pad having two marker plates removably inserted therein;

FIG. 3 is a top elevation view showing the location of a plurality of reference markers within the marker plates inserted within the location pad;

FIG. 4 is a perspective view of coordinate axes relative to a patient table; and

FIG. 5 is a perspective view of a marker rotated from an initial location to a rotated location relative to an X-ray imaging plane.

### DETAILED DESCRIPTION

The following description is merely exemplary in nature and is not intended to limit the present disclosure, application, or uses. It should be understood that throughout the drawings, corresponding reference numerals indicate like or corresponding parts and features.

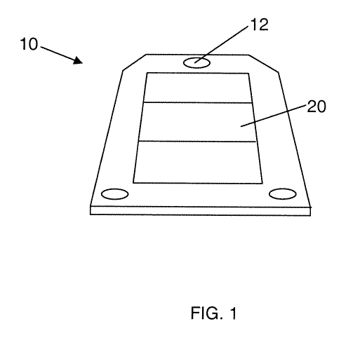

According to various aspects of the present disclosure, there are provided various exemplary embodiments systems and methods for providing registration of a localization system with an imaging system associated with a navigational control system for a medical device. One embodiment of a system according to the principles of the present invention includes an X-ray imaging system, and a localization system having a location pad generally shown at 100 in FIG. 1. The localization system's location pad is preferably capable of sensing electromagnetic signals transmitted and received between the medical device and the location pad, which signals are processed by the localization system for determining the position of the distal end of the medical device within a subject's body. The location pad is positioned relative to a patient table, to provide for monitoring of the location of a medical device that is inserted within a subject's body positioned on the patient table.

The location pad includes at least two plates removably inserted within the location pad, which plates include a plurality of reference markers that are visible by the X-ray imaging system. An imaging system associated with the navigational control system provides for displaying a single X-ray image that includes the plurality of reference markers. A control means is provided that is configured to obtain a registration of the localization system's three-dimensional coordinates for the plurality of reference markers to the two-dimensional coordinates for the plurality of reference markers obtained from the single X-ray image. The control means of the localization system is configured to determine a coordinate transformation that obtains a best fit registration of the localization system's three-dimensional coordinates for the plurality of reference markers, to the two-dimensional coordinates for the plurality of reference markers obtained from the single X-ray image, without requiring any three-dimensional coordinates derived from X-ray images of any marker location. Further aspects relate to methods for obtaining registration of the localization system's three-dimensional coordinates for a plurality of markers having known locations to the two-dimensional coordinates for the plurality of reference markers obtained from the single X-ray image.

In an interventional medical system for directing and manipulating the distal tip of the medical device by remote actuation, a navigation system's coordinates are preferably registered to an imaging system's coordinates. For example, the navigation system may employ external magnetic fields to guide the medical device. One such system is the Niobe/Navigant system manufactured by Stereotaxis. In such a magnetic navigational system, the magnetic field source magnet could be mechanically registered to the imaging system by the use of suitably placed markers placed in known positions with respect to the navigational system. These positions may also be ascribed coordinates in the reference frame of the imaging system since information from multiple imaging projections or from three dimensional imaging would be available from the imaging system. The positions of at least three markers determined in both coordinate systems suffices to generate a rigid coordinate transformation (rotation and translation), which is required for registration of the navigation system with the imaging system. The imaging system may be a fluoroscopic X-ray imaging system for example, which could provide a visual display of one or more reference markers that are radiopaque and visible in one or more X-ray image planes, from which the markers may be ascribed coordinates.

In addition, at least one marker that is radiopaque may be disposed on the distal end of the medical device so as to be visible in one or more X-ray image planes of the imaging system, from which the distal end of the medical device may be ascribed a coordinate location in the reference frame of the imaging system. The marker in the distal end of the medical device can help the physician visualize the catheter and thereby properly position and orient the medical device.

A localization system used for determining and monitoring the location of the distal end of the medical device positioned in a subject's body, to facilitate navigation. Particularly in the accurate movement and positioning of a medical device it is desirable to know the current location and/or orientation of that device. Various localization systems have been developed for this purpose, including magnetic localization systems, which use electromagnetic signals transmitted to or from the medical device to determine the location of the medical device. An example of such a system is the CARTO™ System, available from Biosense-Webster Inc. The localization system utilizes electromagnetic signals transmitted and received between a localization pad and a localization coil disposed within the distal end of the medical device. These signals may be processed to determine the location of the distal end of the medical device relative to the localization pad, within the coordinate system of the localization system. In this manner, the location of the medical device may be monitored by the localization system rather than by repeated use of the Fluoroscopic Imaging system, thereby reducing the exposure to X-rays.

The localization system can also be used to determine the coordinates of reference markers, by using utilizes electromagnetic signals transmitted and received between the localization pad and each individual reference marker. Thus, reference markers placed on the patient table may be located with respect to the localization system, to provide a mutual registration of the distinct coordinate frames employed by the various systems. In practice, such a registration would be performed at the start of a medical procedure.

In one embodiment, a CARTO™ localization system for integration with a Niobe/Navigant magnetic navigational system includes reference markers in the localization system's sensing pad, which provides for improved registration of the CARTO and Niobe coordinate systems. The reference markers may be positioned in known locations with respect to a localization coordinate system, such that the location coordinates of each reference marker do not need to be determined. For example, a plurality of reference markers may be provided on a localization pad at known locations relative to the localization pad/localization system coordinates. It should be noted, however, that the physician may also facilitate registration, for example, through selection of a discrete number of the reference markers included on the localization pad, by pointing to the selected reference markers using a localization wand.

In the first embodiment, the Carto localization system has a plurality of markers embedded in two thin, flat “plates” that can be removably inserted into the localization system's location pad. This mechanical insertion of the plates may be repeated with a high degree of accuracy, with no more than about 0.25 degree orientation variation in placement of the plate within the location pad. The plates themselves have little or no significant radio-opacity, such that a user could optionally keep the location pad markers in place during clinical use without significant obstruction of the X-ray image view. This could alleviate the need to constantly remove and insert the marker plates within the localization pad.

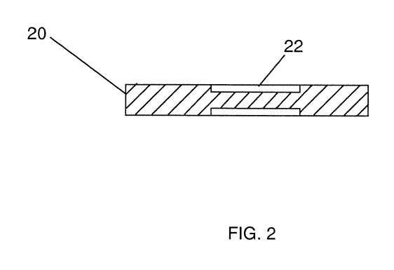

Referring to FIG. 1, one or more flat plates having embedded reference markers are located within the localization pad 110. A first plate 120 may be mounted in a horizontal plane in a central area of the location pad 110, at or near the top surface (closest to the patient table) of the location pad 110. Another plate 124 may be positioned directly (vertically) below the first plate 120 at the bottom surface of the location pad 110. The plates 120 and 124 are configured to be mechanically slipped into the location pad in the positions shown in FIG. 2, with plate 124 being positioned below plate 120. The plates 120 and 124 further comprise a plurality of reference markers embedded therein to provide a distribution or grid of markers on the location pad 110.

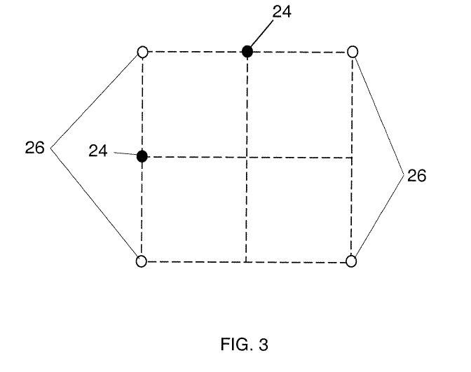

In the embodiment shown in FIG. 3, a total of six radio-opaque markers are provided in the distribution pattern illustrated. The top plate has two markers 130 and 132, and the bottom plate has four markers 134, 136, 138 and 140. The reference markers are radio-opaque, and preferably are made of lead. The markers generally comprise a ball about 2 millimeters (0.08 inches) in diameter, to form nodes of an imaginary flat, square grid.

Markers 130 and 132 shown in FIG. 3 have a center-to-center spacing of about 30 millimeters (1.15 inches) and markers 134-140 have a center-to-center spacing of about 60 millimeters (2.30 inches). It should be noted that both the top and bottom plates 120 and 124 do not have a marker in the center of the imaginary grid arrangement. The top and bottom plates 120 and 124 (and markers 130, 132 and 134-140) are separated by a vertical distance of about 45 millimeters (plane center-to-center). The actual spacing of the marker balls is made to have an accuracy within 75 to 100 microns or better, and the markers are also made co-planar to the same level of accuracy. The grid on the top plate 120 and that of the bottom plate 124 are aligned to within about 0.2-0.3 millimeters. Accordingly, the positions of the grid centers of the top and bottom plates 120 and 124, when slid into the location pad, are known relative to the CARTO localization coordinates to an accuracy of about 0.2 millimeters (0.08 inches). It should be noted that the grid pattern of the plates shown in FIG. 3 are centered on the plates 120 and 124, such that the plates may be inserted with either of their flat sides on top or facing upward.

In an alternate embodiment, the grid pattern is laterally offset towards a patient's left side by 2 centimeters, which may provide for reference markers in known locations that will not interfere with the imaging of a region within the center of the patient. In this alternate construction of plates 120 and 124, the top surface of the plate needs to be clearly marked or identifiable to ensure that the correct side faces up. In any embodiment, the removably insertable plates 120 and 124 are configured to lock into place prior to registration of the CARTO localization system, such that the placement or removal of the marker plate does not disturb the registration in any way.

The registration process utilized to convert the Carto system coordinates to the patient table coordinates employed by Niobe/Navigant magnetic navigational system is described in further detail. In operation, a patient is loaded on the patient table, and a patch is placed on the patient's back. The position of the location pad of the CARTO system is adjusted relative to the particular region of interest on the patient, such as the patient's heart. The location pad is locked into place, preferably with a verifiable audible click sound, or a visible “locked” position. The marker plates 120 and 124 are then inserted into the location pad 110 and locked into place. The C-Arm of an X-ray fluoroscopy imaging system is positioned or placed in an AP view. The patient table position is adjusted until all the markers appear in the live fluoro image display. The Source-to-Image distance is adjusted as needed until all the markers are comfortably within the field of view of the imaging system (i.e.—the markers are all completely visible and clear of the image display edge). The resulting fluoro image is transferred to the Niobe/Navigant system, which displays the image of the region in a display device.

The CARTO system is then registered to the Navigant system. This may be done by the user clicking on a “Register” button in the CARTO-RMT panel (upon which the button may become highlighted). The CARTO system then registers the location pad, and known locations of the reference markers on the location pad, within the coordinate system of the CARTO localization system. Then, on the single AP image transferred to the Navigant system display, the plurality of reference markers (which are radio-opaque and visible in the fluoro image) are “marked” or pointed to by the physician, in any order. The user or physician may then click again on the highlighted “Register” button on the CARTO system. The CARTO system coordinates for the markers are then “registered” to the table coordinate system of the Navigant for the locations of the visible reference markers that were selected or pointed to by the physician. The Navigant system uses the AP image information to perform the registration, and displays a registration error figure on its Carto-RMT panel. The registration error is also sent to Carto for display.

Registration Algorithm

In another aspect of the present disclosure, a method for registering a plurality of reference markers with known coordinates within a localization system with a navigational system is provided, which comprises determining a transformation for converting the two-dimensional coordinates of the marker locations within a single X-ray image plane to the navigational system's frame of reference (table coordinates). The CARTO system coordinates of the six markers are known since the markers are embedded at fixed, known locations in the rigid frame of the Location Pad.

It is important to note that three dimensional Fluoro coordinates of the six markers are not available to the Navigant system, since only a single image is used as input data. Two or more X-ray image views are always required to determine the three dimensional coordinates of an X-ray point, whereas the present methods utilize only a single X-ray view (with the six markers visible in this single view) in the registration scheme employed by the Navigant system. Accordingly, registration is not achieved by determination of the actual three dimensional locations of the X-ray visible markers, which can only be obtained through X-ray images of the markers in at least two orthogonal planes.

The method comprises assigning (a) the two dimensional locations of the six markers in the single X-ray image as xᵢ(i=1, . . . , 6). The method then proceeds to (b) assign the three dimensional CARTO coordinates of the six markers as zᵢ(i=1, . . . , 6). Let R be a three dimensional rotation matrix and t a three dimensional translation vector that together define a rigid transformation to convert Carto coordinates to patient table coordinates. Initially R and t are unknown, and will be determined by an algorithm. Let P be a projective transformation that projects three dimensional points to the (two dimensional) X-ray plate. The projective transformation P is known to Navigant since the C-arm geometry parameters are known.

In the next step (c), the method defines the two dimensional points as:

yᵢ=P(Rzᵢ+t) (1)

and (d) defines the cost function as:

C=(Sum over i)|yᵢ−x_{i|}² (2)

In the next step (e), the method proceeds to the minimization of the cost function C. Note that the cost function is a sum over two dimensional distances. The required rigid transformation (R, t) is found by mathematical minimization of the cost function C.

The rigid transformation determined by the method is the registration transformation that converts CARTO coordinates to patient table coordinates. The method of determining the transformation for providing registration of the CARTO coordinates to the patient table coordinates from a single X-ray image plane will be described in detail below.

Let the grid spacing be L, and let the vertical separation between the two planes be h. The distance D can then be:

D=(h²+L²)^{1/2}

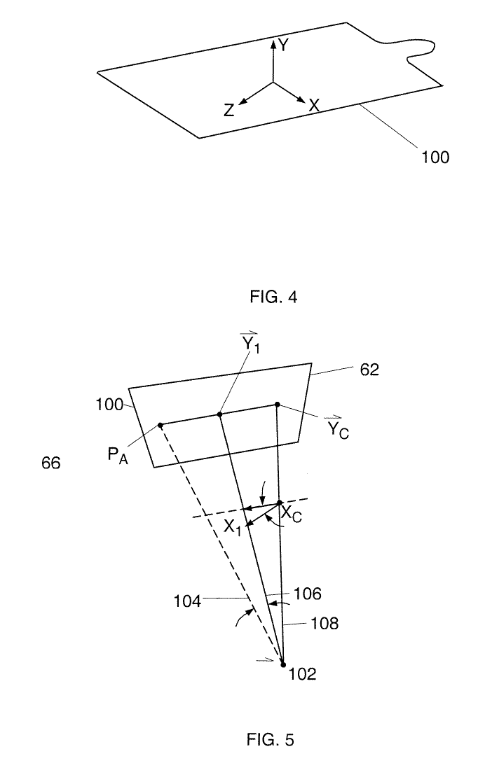

A coordinate axis is selected as shown in FIG. 4. One of the markers, such as marker C, is selected as the origin of the local coordinate system (center of rotation). Let the unit vector \vec{u}₁ be:

$$\begin{matrix}{{u}^{->}}_{1} = ( {{X}^{->}}_{1} - {{X}^{->}}_{C} ) = ( \begin{matrix}- L \\ - h \\ 0\end{matrix} ) , & \\ \text{and} & \\ {{u}^{->}}_{2} = ( {{X}^{->}}_{2} - {{X}^{->}}_{C} ) = ( \begin{matrix}L \\ - h \\ 0\end{matrix} ) , & \\ \text{where} & \\ {{u}^{->}}_{3} = ( {{X}^{->}}_{3} - {{X}^{->}}_{C} ) = ( \begin{matrix}- L \\ 0 \\ L\end{matrix} ) & \end{matrix}$$

in the non-rotated state of the markers (i.e.—the axes of the marker insert are aligned with the coordinate system axes). Let \vec{X}₀=the source location as shown in FIG. 5, which shows the initial position of marker C, \vec{X}_{c}, and the rotated position of marker C, \vec{X}₁′, relative to an image plane. It follows that the rotation ψ₁ causes the projection of marker C on the image plane at point \vec{y}_{c} to shift to \vec{y}₁. (a projective transformation that projects three dimensional points to the two dimensional X-ray plane is known to the Navigant system since the imaging C-arm geometry parameters are known). The point \vec{P}_{A} is the point on the image plane where a line extending perpendicular to the line \vec{y}_{c-\vec{y}}₁ intersects the source location \vec{X}₀. It can be seen that line \vec{P}_{A}−\vec{y}_{c} =x(\vec{y}₁=\vec{y}_{c}). Let the point \vec{P}_{A}=\vec{y}_{c}+x (\vec{y}₁−\vec{y}_{c}), where the product of the perpendicular line and line \vec{Y}_{c}−\vec{y}₁ is (\vec{P}_{A}−\vec{X}₀)·(\vec{y}₁−\vec{y}_{c})=0, Then:

$$⇒ {{P}^{->}}_{A} = {{y}^{->}}_{C} - \frac{⌊ ( {{y}^{->}}_{C} - {{X}^{->}}_{0} ) \cdot ( {{y}^{->}}_{C} - {{y}^{->}}_{1} ) ⌋ ( {{y}^{->}}_{C} - {{y}^{->}}_{1} )}{\vert {{y}^{->}}_{C} - {{y}^{->}}_{1} \vert}$$

$$Cos {ψ}_{1} = \frac{( {{y}^{->}}_{1} - {{X}^{->}}_{0 } ) ( {{P}^{->}}_{A} - {{X}^{->}}_{0} )}{\vert {{y}^{->}}_{1} - {{X}^{->}}_{C} \vert \vert {{P}^{->}}_{A} - {{X}^{->}}_{0} \vert}$$

Next, it must be determined whether the direction of rotation ψ₁ is positive or negative. If the product of (\vec{y}₁−\vec{P}_{A})·(\vec{y}_{c}−\vec{P}_{A})>0 and line (\vec{y}₁−\vec{P}_{A})<line (\vec{y}_{c}−\vec{P}_{A}), then define ε₁₀₄ =−1, else define ε_{ψ}=+1.

$${ψ}_{1} = {ɛ}_{ψ} {Cos}^{- 1} \{ \frac{( {{y}^{->}}_{1} - {{X}^{->}}_{0} ) \cdot ( {{P}^{->}}_{A} - {{X}^{->}}_{0} )}{\vert {{y}^{->}}_{1} - {{X}^{->}}_{0} \vert \vert {{P}^{->}}_{A} - {{X}^{->}}_{0} \vert} \}$$

From the Figure

$$D = \vert {{X}^{->}}_{1}^{′} - {{X}^{->}}_{C} \vert$$

$$Cos {ξ}_{1} = \frac{( {{y}^{->}}_{1} - {{y}^{->}}_{c} ) \cdot ( {{X}^{->}}_{1} - {{X}_{C}}^{->} )}{d \vert {{y}^{->}}_{1} - {{y}^{->}}_{c} \vert}$$

$$\text{Let}$$

$${ɛ}_{1} = \text{sign} \{ ( {{P}^{->}}_{A} - {{X}^{->}}_{0} ) \cdot ( {{X}^{->}}_{1}^{′} - {{X}^{->}}_{C} ) \}$$

$$\text{and} \text{define}$$

$${ξ}_{1} = - {ɛ}_{1} {Cos}^{- 1} \{ \frac{( {{y}^{->}}_{1} - {{y}^{->}}_{c} ) \cdot ( {{X}^{->}}_{1} - {{X}^{->}}_{C} )}{D \vert {{y}^{->}}_{1} - {{y}^{->}}_{c} \vert} \}$$

From the Figure, length A is given by:

A₁=D Cos ξhd 1+D Sin ξ₁ Tan ψ₁

Let D₁=|\vec{X}_{c}−\vec{X}₀|

Then similarity gives where

$$\frac{\vert {{Y}^{->}}_{1} - {{Y}^{->}}_{C} \vert}{{A}_{1}} = \frac{\vert {{y}^{->}}_{C} - {{X}^{->}}_{0} \vert}{{D}_{1}} ,$$

where

$${D}_{1} = \frac{{A}_{1} \vert {{Y}^{->}}_{C} - {{X}^{->}}_{0} \vert}{\vert {{Y}^{->}}_{1} - {{Y}^{->}}_{C} \vert}$$

Rotations

Assume first a rotation of φ the Z-axis, followed by a rotation of φ about the y-axis. The resulting rotation matrix (3×3).

$$M = ( \begin{matrix}{C}_{φ} & 0 & {S}_{φ} \\ 0 & 1 & 0 \\ - {S}_{φ} & 0 & {C}_{θ}\end{matrix} ) ( \begin{matrix}{C}_{φ} & {S}_{φ} & 0 \\ {S}_{φ} & {C}_{φ} & 0 \\ 0 & 0 & 1\end{matrix} ) ,$$

followed by a rotation of

$$= ( \begin{matrix}{C}_{φ} {C}_{ϕ} & - {C}_{φ} {S}_{ϕ} & {S}_{φ} \\ {S}_{ϕ} & {C}_{ϕ} & 0 \\ - {S}_{φ} {C}_{ϕ} & {S}_{φ} {S}_{ϕ} & {C}_{φ}\end{matrix} )$$

takes \vec{u}₁, \vec{u}₂, and \vec{u}₃ to \vec{u}₁′, \vec{u}₂′, and \vec{u}₃′:

$${{u}^{->}}_{1}^{′} = ( \begin{matrix}L Cos φ Cos ϕ + L Cos φ Sin ϕ \\ - L Sin ϕ - L Cos ϕ \\ L Sin φ Cos ϕ - L Sin φ Sin ϕ\end{matrix} )$$

$${{u}^{->}}_{2}^{′} = ( \begin{matrix}L Cos φ Cos ϕ + L Cos φ Sin ϕ \\ L Sin ϕ - L Cos ϕ \\ - L Sin φ Cos ϕ - L Sin φ Sin ϕ\end{matrix} )$$

$${{u}^{->}}_{3}^{′} = ( \begin{matrix}- L Cos φ Cos ϕ + L Sin φ \\ - L Sin ϕ \\ L Sin φ Cos ϕ - L Cos φ\end{matrix} )$$

Finally, assume a rotation of χ about \vec{u}₁′; the corresponding rotation matrix is:

$${R}_{χ} - {I}_{3 \times 3} + \frac{1}{D} Sin χ {U}_{1}^{′} + \frac{1}{{D}^{2}} ( 1 - Cos χ ) {U}_{1}^{′2}$$

$$\text{where}$$

$${U}_{1}^{′} = ( \begin{matrix}0 & - {u}_{1 , z}^{′} & - {u}_{1 , y}^{′} \\ {u}_{1 , z}^{′} & 0 & - {u}_{1 , x}^{′} \\ - {u}_{1 , y}^{′} & {u}_{1 , x}^{′} & 0\end{matrix} )$$

Using the small angle approximation for χ, leads to

$${R}_{χ} = {I}_{3 \times 3} + \frac{χ}{D} {U}_{1}^{′} + \frac{{χ}^{2}}{2 {D}^{2}} {U}_{1}^{′2}$$

The new location of marker is now

$$\begin{matrix}q = {{X}^{->}}_{C} + {R}_{X} {{u}^{->}}_{3}^{′} = ( {x}_{C} + {{u}^{->}}_{3}^{′} ) + \frac{χ}{D} {U}_{1}^{′} {{u}^{->}}_{3}^{′} + \frac{{χ}^{2}}{2 {D}^{2}} {U}_{1}^{′2} {{u}^{->}}_{3}^{′} & \\ \text{Define} & \\ {A}^{->} ≡ ( {{X}^{->}}_{C} + {{u}^{->}}_{3}^{′} ) & \\ {B}^{->} ≡ \frac{1}{D} {U}_{1}^{′} {{u}^{->}}^{′} & \\ {C}^{-> } ≡ \frac{1}{2 D} {U}_{1}^{′} {B}^{->} & \\ \text{so} & \\ q = {A}^{->} + χ {B}^{->} + {χ}^{2} {C}^{->} & \end{matrix}$$

Define the cost function

$${C}_{1} = \vert {q}^{->} - {{X}^{->}}_{0} \vert - ( {q}^{->} - {{X}^{-> }}_{0} ) \cdot {n}^{->}$$

$$\text{where}$$

$${n}^{->} = ( {{y}^{->}}_{3} - {{X}^{->}}_{0} ) = \frac{( {{y}^{->}}_{3} - {{X}^{->}}_{0} )}{\vert {{y}^{->}}_{3} - {{X}^{->}}_{0} \vert}$$

Minimizing C₁ yields the rotation χ that lets \vec{q} “best align” with the projection \vec{y}₃ of marker 3 on the X-ray plane. Let:

\vec{i}=(\vec{A}−\vec{X}₀) and |\vec{l}|

Then

|\vec{q}−\vec{X}₀|²=|\vec{A}−\vec{X}₀|²+χ²B²χ⁴C⁴+2χ\vec{l}·\vec{B}+2 χ³(\vec{b}·\vec{c})+2χ²(\vec{l}·\vec{C})

so that

$$\begin{matrix}\vert {q}^{->} - {{X}^{->}}_{0} \vert = {l [ 1 + 2 χ \frac{( {l}^{->} \cdot {B}^{->} )}{{l}^{2}} + \frac{{χ}^{2}}{{l}^{2}} ( {B}^{2} + 2 l \cdot C ) + 0 ( {χ}^{3} ) ]}^{\frac{1}{2}} \\ = l [ 1 + χ \frac{( {l}^{->} \cdot {B}^{->} )}{{l}^{2}} + \frac{{χ}^{2}}{2 {l}^{2}} ( {B}^{2} + 2 l \cdot C ) - \frac{1}{8} 4 {χ}^{2} \frac{{( {l}^{->} \cdot {B}^{->} )}^{2}}{{l}^{4}} ] \\ = l [ 1 + χ \frac{( {l}^{->} \cdot {B}^{->} )}{{l}^{2}} + {χ}^{2} ( ( {B}^{2} + 2 l \cdot C ) - \frac{{( {l}^{->} \cdot {B}^{->} )}^{2}}{2 {l}^{4}} ) ]\end{matrix}$$

$$( {q}^{->} - {{X}^{->}}_{0} ) \cdot {n}^{->} = {l}^{->} \cdot {n}^{->} + χ ( {B}^{->} \cdot {n}^{->} ) + {χ}^{2} ( {C}^{->} \cdot {n}^{->} )$$

Then minimizing C₁ yields

$${χ}^{*} = - \frac{1}{2} \frac{( \frac{{l}^{->} \cdot {B}^{->}}{l} + {B}^{->} \cdot {n}^{->} )}{[ \frac{( {B}^{2} + 2 {l}^{->} \cdot {C}^{->} )}{2 l} - \frac{{( {l}^{->} \cdot {B}^{->} )}^{2}}{2 {l}^{3}} + {C}^{->} \cdot {n}^{->} ]}$$

If (φ,φ) are known, χ* gives the remaining rotation. We can then construct a d₂ for marker 2 and minimize |d₁−d₂|.

Algorithm

Definitions:

\vec{y}_{c}=coordinates of projection marker C

\vec{y}₁=coordinates of projection marker 1

\vec{y}₂ =coordinates of projection marker 2

\vec{y}₃=coordinates of projection marker 3

\vec{X}₀=coordinates of X-ray source

Define a range −15°<φ<15°, and likewise sub-divide the range into 200 equal intervals. Define \vec{P}_{A} as

$${{P}^{->}}_{A} = {{y}^{->}}_{C} - \frac{[ ( {{y}^{->}}_{C} - {{X}^{->}}_{0} ) \cdot ( {{y}^{->}}_{C} - {{y}^{->}}_{1} ) ] ( {{y}^{->}}_{C} - {{y}^{->}}_{1} )}{\vert {{y}^{->}}_{C} - {{y}^{->}}_{1} \vert}$$

If the product of (\vec{y}₁−\vec{P}_{A})·(\vec{y}_{c}−\vec{P}_{A})>0 and line (\vec{y}₁−\vec{P}_{A})<line (\vec{y}_{c}−\vec{P}_{A}), then define ε₁₀₄ =−1, else define ε_{ψ}=+1.

Define

$${ψ}_{1} = {ɛ}_{ψ 1} {Cos}^{- 1} \{ \frac{( {{y}^{->}}_{1} - {{X}^{->}}_{0} ) \cdot ( {{P}^{->}}_{A} - {{X}^{->}}_{0} )}{\vert {{y}^{->}}_{1} - {{X}^{->}}_{0} \vert \vert {{P}^{->}}_{A} - {{X}^{->}}_{0} \vert} \}$$

Define

$${{P}^{->}}_{B} = {{y}^{->}}_{C} - \frac{[ ( {{y}^{->}}_{C} - {{X}^{->}}_{0} ) \cdot ( {{y}^{->}}_{C} - {{y}^{->}}_{2} ) ] ( {{y}^{->}}_{C} - {{y}^{->}}_{2} )}{\vert {{y}^{->}}_{C} - {{y}^{->}}_{2} \vert}$$

If the product of (\vec{y}₂−\vec{P}_{B})·(\vec{y}_{c}−\vec{P}_{B})>0 and line (\vec{y}₂−\vec{P}_{B})<line (\vec{y}_{c}−\vec{P}_{B}), then define ε_{ψ}=−1, else define ε_{ψ}=+1.

Define

$${ψ}_{2} = {ɛ}_{ψ2} {Cos}^{- 1} \{ \frac{( {{y}^{->}}_{2} - {{X}^{->}}_{0} ) \cdot ( {{P}^{->}}_{B} - {{X}^{->}}_{0} )}{\vert {{y}^{->}}_{2} - {{X}^{->}}_{0} \vert \vert {{P}^{->}}_{B} - {{X}^{->}}_{0} \vert} \}$$

For every (φ, φ) in the chosen ranges, initialize C₂=0, so that C₂ is essentially a 200×200 matrix.

$${{u}^{->}}_{1}^{′} = ( \begin{matrix}L Cos φCosϕ + L Cos φSinϕ \\ - L Sin ϕ - L Cos ϕ \\ L Sin φ Cos ϕ - L Sin φ Sin ϕ\end{matrix} )$$

$${{u}^{->}}_{2}^{′} = ( \begin{matrix}L Cos φCosϕ + L Cos φSinϕ \\ L Sin ϕ - L Cos ϕ \\ - L Sin φ Cos ϕ - L Sin φ Sin ϕ\end{matrix} )$$

$${{u}^{->}}_{3}^{′} = ( \begin{matrix}- L Cos ϕCosϕ + L Sin ϕ \\ - L Sin ϕ \\ L Sin φ Cos ϕ + L Cos φ\end{matrix} )$$

$$\text{Define}$$

$${ɛ}_{1} = \text{sign} \{ ( {{P}^{->}}_{A} - {{X}^{->}}_{0} ) \cdot ( {{X}^{->}}_{1}^{′} - {{X}^{->}}_{C} ) \} , \\ \text{and}$$

$${ξ}_{1} = - {ɛ}_{1} {Cos}^{- 1} \{ \frac{( {{y}^{->}}_{1} - {{y}^{->}}_{c} ) \cdot ( {{X}^{->}}_{1} - {{X}^{->}}_{C} )}{D \vert {{y}^{->}}_{1} - {{y}^{->}}_{c} \vert} \}$$

Define A₁=D Cos ξ₁+D Sin ξ₁ Tan ψ₁, and

$${D}_{1} = \frac{{A}_{1} \vert {{Y}^{->}}_{C} - {{X}^{->}}_{0} \vert}{\vert {{Y}^{->}}_{1} - {{Y}^{->}}_{C} \vert}$$

If D₁>|\vec{Y}_{C}−\vec{X}₀|, then set C₂=10⁶ and break;

Define

$${A}^{->} ≡ ( {{X}^{->}}_{C} + {{u}^{->}}_{3}^{′} )$$

$${B}^{->} ≡ \frac{1}{D} {U}_{1}^{′} {{u}^{->}}^{′}$$

$${C}^{->} ≡ \frac{1}{2 D} {U}_{1}^{′} {B}^{->}$$

$${l}^{->} = ( {A}^{->} - {{X}^{->}}_{0} )$$

$$l = \vert {l}^{->} \vert$$

$${n}^{->} = ( {{y}^{->}}_{3} - {{X}^{->}}_{0} ) = \frac{( {{y}^{->}}_{3} - {{X}^{->}}_{0} )}{\vert {{y}^{->}}_{3} - {{X}^{->}}_{0} \vert}$$

Define and record

$${χ}^{*} = - \frac{1}{2} \frac{( \frac{{l}^{->} \cdot {B}^{->}}{l} + {B}^{->} \cdot {n}^{->} )}{[ \frac{( {B}^{2} + 2 {l}^{->} \cdot {C}^{->} )}{2 l} - \frac{{( {l}^{->} \cdot {B}^{->} )}^{2}}{2 {l}^{3}} + {C}^{->} \cdot {n}^{->} ]}$$

If χ*>0.3, then set C₂=10⁶ and break; define the 3×3 matrix (rotation) as:

$${R}_{χ} = {I}_{3 \times 3} + \frac{1}{D} Sin χ {U}_{1}^{′} + \frac{1}{{D}^{2}} ( 1 - Cos χ ) {U}_{1}^{′2}$$

$$\text{where}$$

$${U}_{1}^{′} = ( \begin{matrix}0 & - {u}_{1 , z}^{′} & - {u}_{1 , y}^{′} \\ {u}_{1 , z}^{′} & 0 & - {u}_{1 , x}^{′} \\ - {u}_{1 , y}^{′} & {u}_{1 , y}^{′} & 0\end{matrix} )$$

Define \vec{u}′=\vec{R}\vec{u}₁, ε₂=sign {(\vec{P}_{B}−\vec{X}₀)·\vec{u}₁′}, and

$${ξ}_{2} = - {ɛ}_{2} {Cos}^{- 1} \{ \frac{( {{y}^{->}}_{2} - {{y}^{->}}_{c} ) \cdot {u}^{{->}_{2}^{″}}}{D \vert {{y}^{->}}_{2} - {{y}^{->}}_{c} \vert} \}$$

Define A₂=D Cos ξ₂+D Sin ξ₂ Tan ψ₂ , and

$${D}_{2} = \frac{{A}_{2} \vert {{Y}^{->}}_{C} - {{X}^{->}}_{0} \vert}{\vert {{Y}^{->}}_{2} - {{Y}^{->}}_{C} \vert}$$

C₂=|D₁−D₂|

Find (φ, φ) such that C₂ is minimized among all values examined; correspondingly, let the X₁ value be \vec{X}, and the D₁ value be \vec{D}. Define:

$$M = ( \begin{matrix}Cos {φ}^{*} Cos {ϕ}^{*} & - Cos {φ}^{*} Sin {ϕ}^{*} & Sin {φ}^{*} \\ Sin {ϕ}^{*} & Cos {ϕ}^{*} & 0 \\ - Sin {ϕ}^{*} Cos {ϕ}^{*} & Sin {φ}^{*} Sin {ϕ}^{*} & Cos {φ}^{*}\end{matrix} )$$

$${{u}^{->}}_{1}^{′ *} = M ( \begin{matrix}- L \\ - h \\ 0\end{matrix} )$$

$${R}_{χ} = {I}_{3 \times 3} + \frac{1}{D} Sin {χU}_{1}^{′ *} + \frac{1}{{D}^{2}} ( 1 - Cos χ ) {( {U}_{1}^{′ *} )}^{2}$$

Here, we define the matrix

$${U}_{1}^{′ *} = ( \begin{matrix}0 & - {u}_{1 , z}^{′ *} & {-}_{1 , y}^{′ *} \\ {u}_{1 , z}^{′} & 0 & - {u}_{1 , z}^{′ *} \\ - {u}_{i , y}^{′ *} & {u}_{1 , x}^{′ *} & 0\end{matrix} )$$

Define the rotation matrix

N=\vec{R}M

$${{x}^{->}}_{C}^{′} = {{D}^{->}}_{1} \frac{( {{y}^{->}}_{C} - {{X}^{->}}_{0} )}{\vert {{y}^{->}}_{C} - {{X}^{->}}_{0} \vert} + {{X}^{->}}_{0}$$

Given a Carto point \vec{P}′:

(i) Swap/Coordinates to account for reflect coordinates axes definition; call resulting point \vec{P}.

(ii) Let \vec{P}_{c,)} be the location of marker C in Carto coordinates, with suitable axes swaps.

(iii) \vec{z}=N(\vec{P}−\vec{P}_{c,0})+\vec{X}_{c} yields the Carto point in table coordinates.

Upon completion of the registration of the localization system and imaging and navigational system, the location of the distal end of the medical device may be determined within both coordinate systems. The CARTO localization system is capable of determining the location of the distal end of the medical device by processing electromagnetic signals transmitted and received between the medical device and the location pad of the localization system. The user can define a location on the Niobe/Navigant workstation with a mouse or other pointing device, to identify the desired location to position the medical device. The Navigant workstation computes the required fields and gradients required to navigate the distal end of the medical device to the new location.

In addition, Anatomical landmarks or markers fixed to the patient can be used to register a local reference frame, and to serve as references for localization. In some situations it may be useful for the physician to facilitate registration, for example by accessing anatomical landmarks within the patient with a localized medical device that is seen both on a fluoroscopic imaging system and in the localization system display. It may be necessary for the physician to facilitate registration, for example by pointing to anatomical landmarks on the patient with a localization wand. When the patient cannot be adequately immobilized, patient localization means may be required to account for his movements during the procedure, and automatically adjust the coordinate registration.

The markers 132-140 are preferably be radio-opaque where the catheter is localized with fluoroscopy. Alternatively, the markers may also comprise ultrasonic transducers, where the catheter is localized using ultrasonic time-of-flight. Likewise, the markers may comprise electromagnetic means, where the catheter is localized using electromagnetic field sensing; or some other type of marker for localizing the catheter.

The advantages of the above described embodiments and improvements should be readily apparent to one skilled in the art, as to enabling registration of imaging systems with respect to navigation systems for medical devices. Additional design considerations may be incorporated without departing from the spirit and scope of the invention. The description in this disclosure is merely exemplary in nature and, thus, variations are not to be regarded as a departure from the spirit and scope of the disclosure. Accordingly, it is not intended that the invention be limited by the particular embodiments or forms described above, but by the appended claims.

What is claimed is:

1. A system for providing registration of a navigational system with localization of a medical device, comprising:
a localization system having a location pad;
at least two plates removably inserted within the location pad, which plates include a plurality of reference markers that are visible to an X-ray imaging device,
a display device for displaying a single X-ray image including the plurality of reference markers; and
a control means configured to obtain a registration of the localization system's three-dimensional coordinates to the two-dimensional coordinates obtained from the single X-ray image.

2. The apparatus of claim 1 wherein the control means is configure to determine a coordinate transformation that obtains a best fit registration of the localization system's three-dimensional coordinates for the plurality of reference markers, to the two-dimensional coordinates for the plurality of reference markers obtained from the single X-ray image, without requiring any three-dimensional coordinates derived from X-ray images of any marker location.

3. The apparatus of claim 1 wherein the three-dimensional coordinates of the locations of a plurality of reference markers within the localization coordinate system.

4. The apparatus of claim 1 wherein the plurality of reference markers form a grid pattern across the location pad.

5. The apparatus of claim 1 wherein the plurality of reference markers are radio-opaque.

6. The apparatus of claim 1 wherein the plurality of reference markers comprise at least three reference markers.

7. The apparatus of claim 1 wherein the location pad is capable of sensing electromagnetic signals transmitted and received between the medical device and the location pad, which signals are processed by the localization system to determine the position of the distal end of the medical device within a subject's body.

8. A method of registering a localization system, having a plurality of reference markers with known coordinates within the localization system, with a navigational system, the method comprising:
determining the three-dimensional coordinates of the locations of a plurality of reference markers within the localization coordinate system;
providing a single two-dimensional X-ray image that includes a visual indication of the plurality of markers;
determining a coordinate transformation for obtaining a best fit registration of the localization system's three-dimensional coordinates to the two-dimensional coordinates obtained from the single X-ray image, for at least one reference marker location.

9. The method of claim 8 further comprising the step of using the transformation to obtain registration of the localization system to the coordinate system utilized by the navigational system.

10. The method of claim 8, wherein the step of determining the best fit registration comprises minimizing a cost function that yields a rotation matrix that lets the transformed two-dimensional location of a marker best align with the two-dimensional projection of the marker's location on the X-ray plane.

11. The method of claim 8, wherein the step of determining the best fit registration comprises determining a three-dimensional rotation matrix and translation vector for converting the localization coordinates for a plurality of marker locations to navigational system coordinates which, when converted by a projection transformation to two-dimensional coordinates of the X-ray plane, yields a minimization of the sum of two-dimensional distances between the transformed two-dimensional coordinates and the two-dimensional coordinates of the plurality of markers derived from the single X-ray image.

12. The method of claim 8, wherein the plurality of reference markers includes as least three reference marker locations.

13. The method of claim 8 wherein the step of determining a coordinate transformation does not require determining the three-dimensional coordinates of a marker location derived from X-ray images of the marker.

14. A method of registering a localization system, having a plurality of reference markers with known coordinates within the localization system, with a navigational system, the method comprising:
determining the three-dimensional coordinates of the locations of a plurality of reference markers within the localization coordinate system;
providing a single two-dimensional X-ray image that provides a two-dimensional location of the plurality of markers;
determining a coordinate transformation for obtaining a best fit registration of the localization system's three-dimensional coordinates for the plurality of reference markers to the two-dimensional coordinates obtained from the single X-ray image for the locations of the plurality of reference markers.

15. The method of claim 14 further comprising the step of using the transformation to obtain registration of the localization system to the coordinate system utilized by the navigational system.

16. The method of claim 14, wherein the step of determining the best fit registration comprises minimizing a cost function that yields a rotation matrix that lets the transformed two-dimensional location of a marker best align with the two-dimensional projection of the marker's location on the X-ray plane.

17. The method of claim 14, wherein the step of determining the best fit registration comprises determining a three-dimensional rotation matrix and translation vector for converting the localization coordinates for a plurality of marker locations to navigational system coordinates which, when converted by a projection transformation to two-dimensional coordinates of the X-ray plane, yields a minimization of the sum of two-dimensional distances between the transformed two-dimensional coordinates and the two-dimensional coordinates of the plurality of markers derived from the single X-ray image.

18. The method of claim 14 further comprising the step of using the transformation to obtain registration of the localization system to the coordinate system utilized by the navigational system.

19. The method of claim 14, wherein the plurality of reference markers includes as least three reference marker locations.

20. The method of claim 14 wherein the step of determining a coordinate transformation does not require determining the three-dimensional coordinates of a marker location derived from X-ray images of the marker.
